(19) United States
(12) Patent Application Publication
(10) Pub. No.: US 2007/0239153 A1
(43) Pub. Date: Oct. 11, 2007

(54) Computer assisted surgery system using alternative energy technology

(76) Inventors: Robert A. Hodorek, Warsaw, IN (US); Donald M. Patmore, Winona Lake, IN (US)

Correspondence Address: ZIMMER TECHNOLOGY - BAKER & DANIELS, 111 EAST WAYNE STREET, SUITE 800, FORT WAYNE, IN, 46802, US

(21) Appl. No.: 11/359,068

(22) Filed: Feb. 22, 2006

(51) Int. Cl. A61B 18/18

(52) U.S. Cl. 606/41

(57) ABSTRACT

A method and apparatus for a computer assisted surgery (CAS) system using alternative energy tissue and bone alteration technology. The CAS system utilizes alternative energy technology which is a directed to a surgical instrument including an alteration or cutting tip. The tip may be in contact with the tissue or bone, or, alternatively, the tip may be distant from the tissue or bone and the energy is projected to the desired cut or alteration site. The CAS system recognizes the location of the tip relative to a desired alteration location or area and de-energizes or varies the energy level when the tip moves away from or out of the predetermined alteration location or path. The CAS system provides a method for altering or resecting bone, for example, in preparation for a prosthetic implant, or a method for altering tissue, for example, cauterizing blood vessels or bonding ligaments to bones.

### BACKGROUND

1. Field of the Invention.

The present invention relates to computer assisted surgery. More particularly, the present invention relates to a method and apparatus for using alternative energy technology which is controlled by a computer assisted surgery system to modify or alter tissues or bones.

2. Description of the Related Art.

Orthopedic implants are commonly used to replace some or all of a patient's joints in order to restore the use of the joints, or to increase the use of the joints, following deterioration due to aging or illness, or injury due to trauma. Accurate altering and resections of bone and soft tissue, such as ligaments, are critical to ensure a proper fit of the orthopedic implants. In a typical joint replacement procedure, a surgeon may employ a computer assisted surgery (CAS) system to facilitate accuracy and precision of the outcome of the procedure.

CAS systems and procedures have been developed for positioning surgical instruments in a predefined position and orientation relative to a patient's anatomical structures. Computer assisted guidance of surgical instruments can be used in orthopedic surgical procedures, for example, to position a cutting instrument in a predefined position and orientation with respect to a bone when preparing the bone to receive a prosthetic implant such as a component of an artificial joint, or to position an alteration instrument in a predefined position and orientation with respect to tissue when cauterizing blood vessels or bonding ligaments to bones. Guidance techniques typically involve acquiring preoperative images of the relevant anatomical structures and generating a database which represents a three-dimensional model of the anatomical structures. The surgical instruments typically have a fixed geometry which is used to create geometric models of the instruments. The geometric models of the instruments can then be superimposed on the model of the relevant anatomical structures.

During the surgical procedure, the position of the instrument(s) being used and the patient's anatomical structures are registered with the anatomical coordinate system of the computer model of the relevant anatomical structures. Registration is the process of defining the geometric relationship between the physical world and a computer model. Registration of the patient with the computer model allows the computer to manipulate the computer model to match the relative positions of various components of the patient's anatomical structure in the physical world. Registration of the instrument(s) used with the computer model allows the computer to display and/or direct the placement of the instrument(s) and prosthetic components relative to the patient's anatomical structure. To assist the registration process, fiducial pins or markers are placed in contact with a portion of the anatomical structure and/or instrument which are also locatable in the computer model. The markers are locatable in space by the computer, thereby providing a geometric relationship between the model and physical anatomical structure. A graphical display showing the relative positions of the instrument and anatomical structures can then be computed in real time and displayed to assist the surgeon in properly positioning and manipulating the surgical instrument with respect to the relevant anatomical structure. Examples of various computer-assisted navigation systems are described in U.S. Pat. Nos. 5,682,886; 5,921,992; 6,096,050; 6,348,058; 6,434,507; 6,450,978; 6,470,207; 6,490,467; and 6,491,699, the disclosures of which are hereby explicitly incorporated herein by reference.

CAS systems typically use a mechanical instrument, such as a rotating drill bit or an oscillating saw blade, to perform bone resection or soft tissue alteration. Some CAS systems are equipped with the ability to recognize the location of the instrument, and allow supply of electrical power to the mechanical instrument when the instrument is in a desired location on or near the body of the patient. The CAS system tracks the movement of the instrument to allow the CAS system to determine whether the instrument is in the desired location. If, for some reason, the instrument moves outside the desired location for alteration of the bone or tissue, the CAS system is able to sense the location and terminate supply of electrical power to the instrument. However, conventional mechanical instruments in CAS systems require a time delay before all mechanical motion of the instrument is completely stopped. For example, after electrical power is removed from a mechanical drill bit, the drill bit may continue to rotate while decelerating. Also, for example, after electrical power is removed from an oscillating saw blade, the blade may continue to oscillate until it comes to a complete stop. An example of such a prior art CAS system which provides guidance to cut a predetermined cut plane includes cutting instrument 15, shown in FIG. 1. Robot arm 17 of a known robotic CAS system may be used to position cut guide 16 in order to make a cut along proximal tibial cut plane 18 on tibia 38 and/or other cut planes using cutting instrument 15. Computer 23 (FIG. 2) may be preprogrammed with the geometry of cut guide 16 and robot ann 17 in order to accurately position blade slot 19 and properly locate proximal tibial cut plane 18.

### SUMMARY

The present invention provides a method and apparatus for a computer assisted surgery (CAS) system using alternative energy tissue and bone alteration technology. The CAS system utilizes alternative energy technology which is a directed to a surgical instrument including an alteration or cutting tip. The tip may be in contact with the tissue or bone, or, alternatively, the tip may be distant from the tissue or bone and the energy is projected to the desired cut or alteration site. The CAS system recognizes the location of the tip relative to a desired alteration location or area and de-energizes or varies the energy level when the tip moves away from or out of the predetermined alteration location or path. The CAS system provides a method for altering or resecting bone, for example, in preparation for a prosthetic implant, or a method for altering tissue, for example, cauterizing blood vessels or bonding ligaments to bones.

In one form thereof, the present invention provides a method for altering an anatomical structure of a patient using a computer assisted surgery system including a computer and an alternative energy source, the method including the steps of registering the anatomical structure of the patient with the computer; inputting into the computer a workspace associated with the anatomical structure of the patient; applying energy from the alternative energy source to the workspace with a surgical instrument; and terminating immediately the application of energy under control from the computer when the surgical instrument deviates from the workspace.

In another form thereof, the present invention provides a computer assisted surgery system for altering an anatomical structure of a patient, the system including a computer including a workspace storage memory storing an identified workspace associated with at least one anatomical structure of a patient; an alternative energy source; a surgical instrument connected to the alternative energy source, the instrument convertible between a first, non-enabled condition associated with the instrument not being present in the workspace in which energy is not supplied to the instrument from the alternative energy source, and a second, enabled condition associated with the instrument being present in the workspace in which energy is supplied to the instrument from the alternative energy source; and an energy source controller associated with the computer, the controller controlling conversion of the instrument from the second, enabled condition to the first, non-enabled condition to immediately terminate energy supplied to the instrument.

In yet another form thereof, the present invention provides a computer assisted surgery system for altering an anatomical structure of a patient, the system controlling an alternative energy source, the system including a computer; means for registering the anatomical structure of the patient with the computer; means for identifying a workspace associated with the anatomical structure; means for applying energy from the alternative energy source to the workspace; and means for immediately terminating a supply of energy from the alternative energy source under control from the computer when the applying energy means deviates from the workspace.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a perspective view of a surgical instrument and a computer navigation device of a known computer assisted surgery (CAS) system;

FIG. 2 is a perspective view of an operating room arrangement including a CAS system according to one embodiment, further showing a patient;

FIG. 3 is a block schematic diagram of the CAS system of FIG. 2;

FIG. 4 is a perspective view of a typical knee joint of a human patient, further illustrating several resection areas and several tissue alteration areas;

FIG. 5 is a perspective view of a surgical instrument attached to an alternative energy source and the computer of the CAS system of FIG. 2;

FIG. 6 is a perspective view of the surgical instrument of FIG. 5, further illustrating the surgical instrument controlled by a robot arm;

FIG. 7 is a perspective view of the surgical instrument of FIG. 5, further illustrating the surgical instrument manually controlled by the hand of a surgeon; and

FIG. 8 is a flow chart of a method according to one embodiment of the present invention.

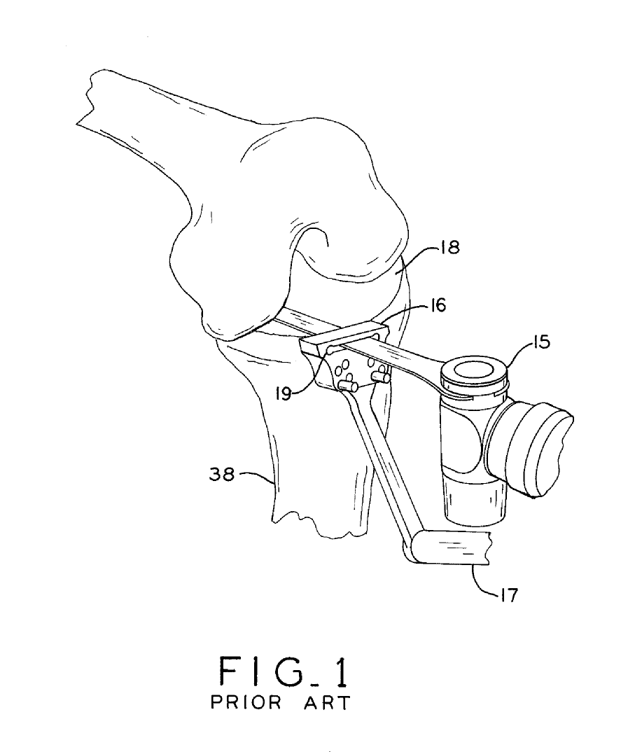

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

The embodiments disclosed below are not intended to be exhaustive or limit the invention to the precise forms disclosed in the following detailed description. Rather, the embodiments are chosen and described so that others skilled in the art may utilize their teachings.

The present invention provides a method and apparatus for a computer assisted surgery (CAS) system using alternative energy tissue and bone alteration technology. The CAS system utilizes alternative energy technology which is a directed to a surgical instrument including an alteration or cutting tip. The tip may be in contact with the tissue or bone, or, alternatively, the tip may be distant from the tissue or bone and the energy is projected to the desired cut or alteration site. The CAS system recognizes the location of the tip relative to a desired alteration location or area and de-energizes or varies the energy level when the tip moves away from or out of the predetermined alteration location or path. The CAS system provides a method for altering or resecting bone, for example, in preparation for a prosthetic implant, or a method for altering tissue, for example, cauterizing blood vessels or bonding ligaments to bones.

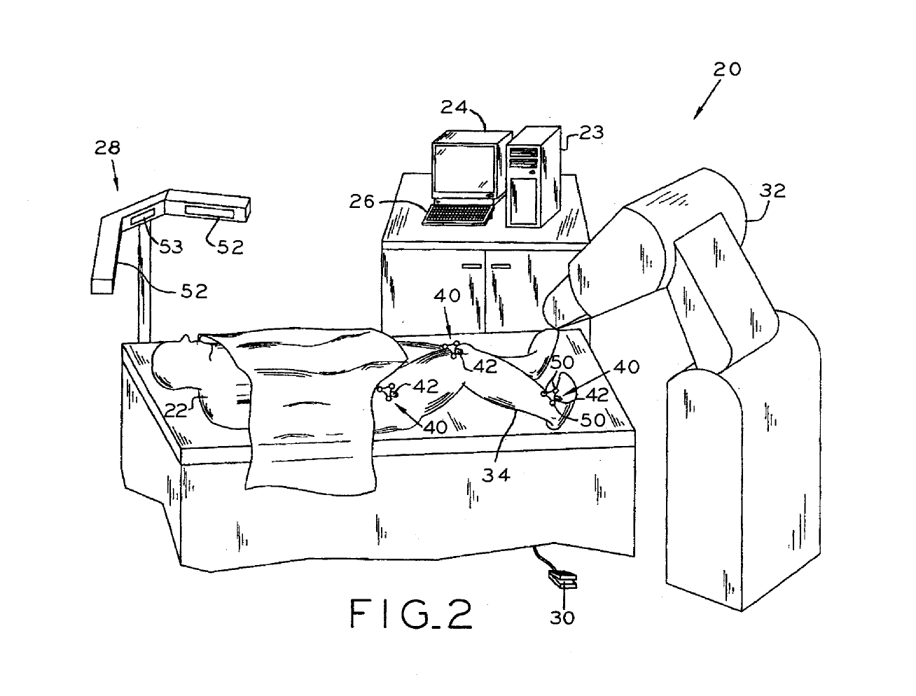

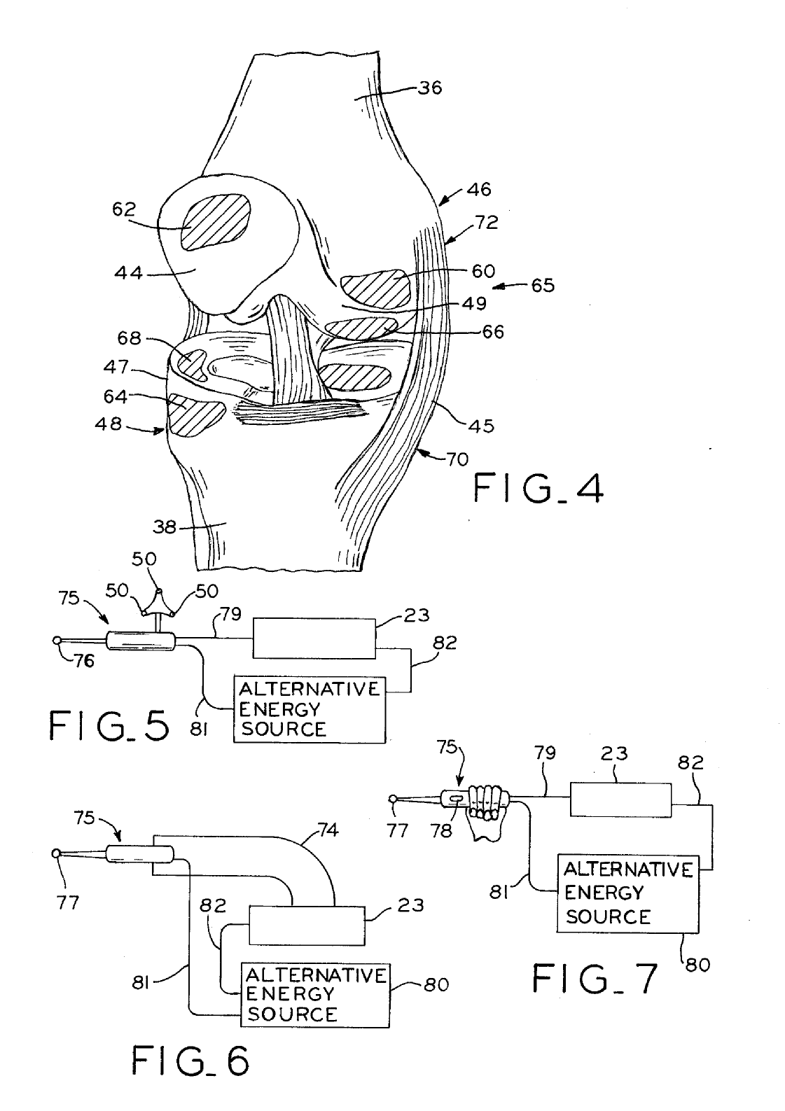

Referring to FIG. 2, an operating room arrangement is shown including computer assisted surgery (CAS) system 20 for aiding surgical procedures performed on patient 22. As described herein, CAS system 20 may be used to provide graphical and other data information relating to the anatomical structures of patient 22 and to provide control to a surgical instrument used to alter tissue or bone in patient 22. CAS system 20 may include computer 23, display 24, keyboard 26, navigation sensor 28, input device 30, and imaging device 32. Generally, computer 23 and navigation sensor 28 determine the position of anatomical structures of patient 22, for example, the position of limb 34, including femur 36 (FIG. 4) and tibia 38 (FIG. 4), may be determined. Navigation sensor 28 detects the position of the anatomical structures by sensing the position and orientation of markers such as reference arrays 40 associated with the anatomical structures. Each reference array 40 may include probe 42 extending through an incision in limb 34 and contacting a bone landmark, for example patella 44 (FIG. 4), distal femur 46 (FIG. 4), and/or proximal tibia 48 (FIG. 4). Each reference array 40 includes an array of reference devices 50 which passively or actively transmit an optical, electromagnetic, or other signal to sensors 52 of navigation sensor 28. If a passive reference device 50 is used, emitter 53 transmits a signal that is reflected by reference device 50 and then received by sensors 52 upon reflection from reference device 50. If an active reference device 50 is utilized, reference device 50 itself generates a signal for transmission to, and detection by, sensors 52.

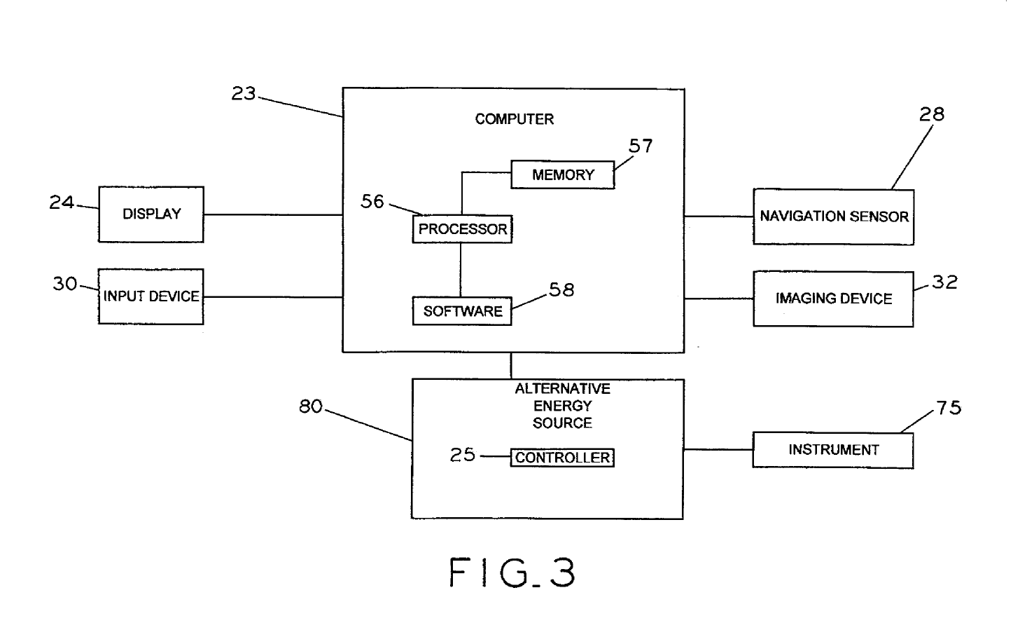

Computer 23, shown in FIGS. 2 and 3, includes processor 56, memory 57, and software 58. Software 58 provides tracking of reference arrays 40 so that graphical and data representations of the anatomical structures of patient 22 may be provided on display 24. To enhance the displayed image and to provide a three-dimensional model of the anatomical structures, imaging device 32 may be used for providing images of the anatomical structures to computer 23. Imaging device 32 may be any of several well-known devices utilized for providing images of internal body structures, such as a fluoroscopic imaging device, a computerized tomography (CT) imaging device, a magnetic resonance imaging (MRI) device, an ultrasound imaging device, a diffraction enhanced imaging (DEI) device, or a positron emission tomography (PET) device.

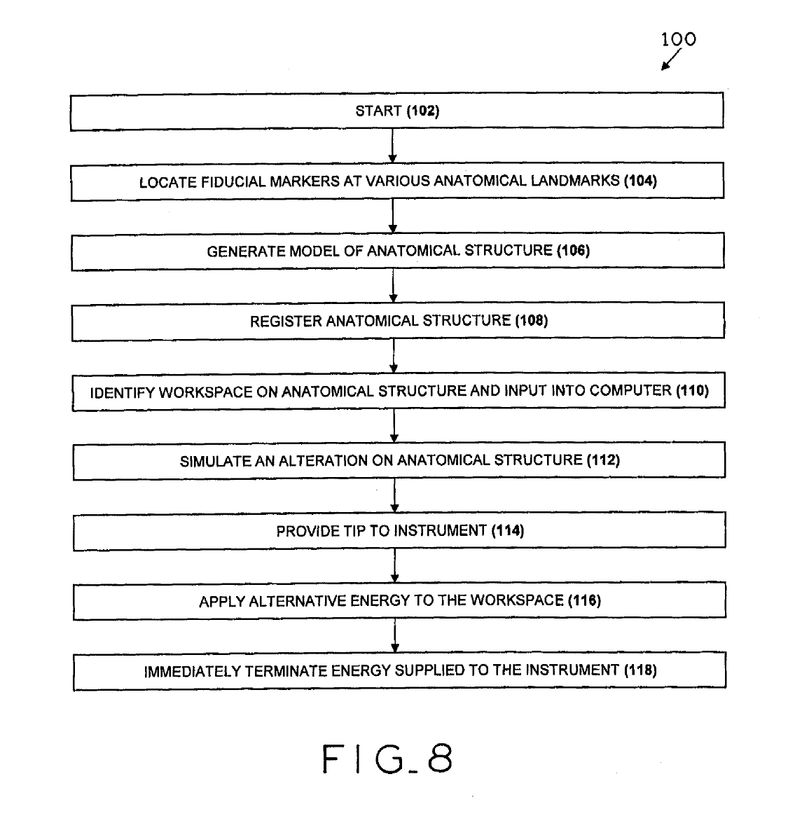

In one embodiment, method 100, shown in FIG. 8, begins at step 102 and may be performed preoperatively or intraoperatively. Method 100 includes steps that, at least in part, may be implemented by software 58 and other components of CAS system 20. Certain steps may also require activity from a surgeon or other assistant.

In step 104, reference arrays 40 (FIG. 2) are located at various bone landmarks of limb 34 (FIG. 2), for example and as shown in FIG. 4, patella 44, distal femur 46, and/or proximal tibia 48 may be located and marked by reference arrays 40. As described previously and referring to FIG. 2, reference arrays 40 may include reference devices 50 which are tracked by navigation sensor 28. Reference array 40 may also include probe 42 which extends through an incision in limb 34 and contacts the desired bone landmarks. Alternatively, the bone landmarks may be located by reference devices 50 which do not penetrate limb 34 and are positioned securely relative to limb 34 by other surgical instrumentation.

In step 106, imaging device 32 (FIG. 2) may be used to provide images of the anatomical structures to computer 23. In one embodiment, multiple fluoroscopic images may be used to construct three-dimensional images of the appropriate anatomical structures. Alternatively, images from CT imaging devices, a combination of fluoroscopic and CT imaging devices, MRI devices, ultrasound imaging devices, DEI devices, or PET devices may be used. Display 24 shows the images of the corresponding anatomical structures.

In step 108, the relevant anatomical structures are registered with CAS system 20. Specifically, the combination of data available from reference devices 50 and images of the anatomical structures form a model of the anatomical structure, for example, knee joint 65 shown in FIG. 4. The model may be further developed by specifying additional landmarks of the anatomical structures which are visible in display 24. The resulting three-dimensional model and images may be overlaid together and used to provide accurate display and simulation of the anatomical structures.

In step 110, a desired workspace is identified and input into memory 57 of computer 23. For the purposes of this document, workspace may be defined as any alteration location, area, or volume, for example, a cutting plane, a drilling axis, a bonding location, a cauterizing location, a resection area, a resection volume, etc. The alteration area may be a desired cutting plane, a drilling axis, a cauterizing location, a bonding location, or any other bone or tissue alteration location, area, or three-dimensional volume. The alteration area may be selected or identified by the surgeon using the information provided from CAS system 20. For example and referring to FIG. 4, the surgeon may virtually select workspace 60 on a condyle of distal femur 46 by defining workspace 60 on computer 23 via keyboard 26, display 24, or any other input means, for example, with a digital pen which the surgeon uses on display 24 to outline the desired alteration area on an anatomical structure. Workspace 60 may be identified to correct, for example, a varus or valgus defect of knee joint 65. Alternatively, the surgeon may virtually select workspace 62 on patella 44 or workspace 64 on proximal tibia 48. Also, the surgeon may virtually select workspace 66 on articular cartilage 49 or workspace 68 on meniscus 47. Also, the surgeon may virtually select workspace 70 or 72 on medial collateral ligament 45 to bond ligament 45 to a bone, e.g., femur 36 or tibia 38, to correct for laxity in ligament 45. The surgeon may also select any other desired alteration, resection, bonding, or cauterizing location for a particular application. Advantageously, the volume, area, location, etc. of workspace 60 having an infinite number of sizes and/or shapes may be manually determined with a probe, e.g., hand drawn around the localized surgical area, and then a depth of workspace 60 may be assigned with computer 23 without being confined to preset orientations and depths dictated by mechanical instruments.

Also, workspace 60 may be advantageously limited to a preset array of implant sizes. For example, the surgeon may input into computer 23 known characteristics of an actual implant to be used in the surgical procedure. Computer 23 may then determine the desired size for workspace 60 based on the known characteristics of the implant. Thus, computer 23 may tailor the size of workspace 60. In one embodiment, computer 23 may set either a minimum size or maximum size of workspace 60 and the actual final size of workspace 60 is determined by the discretion of the surgeon.

Although described hereinafter with respect to workspace 60 of a knee joint, the present method is equally applicable to any desired resection, alteration, bonding, or cauterizing location, area, or three-dimensional volume, or any other bone or tissue modification location, area, or three-dimensional volume.

In another embodiment, the surgeon identifies and selects the alteration area using a probe without any prior assistance from CAS system 20, i.e., there is no imaging involved. However, imaging of the anatomical structures of patient 22 may also be used when the surgeon identifies the alteration location, area, or volume using a probe. Referring now to FIGS. 4 and 5, probe or surgical instrument 75 may be used to trace out a perimeter around a defective portion of the bone to define, for example, workspace 60 on distal femur 46. Instrument 75 may include a plurality of reference devices 50 or other known geometry identifiers which communicate positional information of instrument 75 to CAS system 20. CAS system 20 can monitor and/or identify the position of distal tip 76 of instrument 75 based on the detected location of reference devices 50 and the known geometry of instrument 75. The surgeon maneuvers instrument 75 such that distal tip 76 contacts distal femur 46 at workspace 60. The surgeon may outline workspace 60 and software 58 may be used to “paint”, i.e., survey, fill in, and/or complete, the remainder of workspace 60 based on actual knowledge of the anatomical structure or based on a generic model of the anatomical structure via extrapolation from the contact points of distal tip 76 with distal femur 46, or, the surgeon may use instrument 75 to identify, i.e., “paint”, fill in, or survey, the entire surface of workspace 60, for example, by contacting distal tip 76 on distal femur 46 in a sweeping or surveying manner across the entire area of workspace 60 in a manner analogous to painting a surface area with a paintbrush. CAS system 20 may also allow the surgeon to input a desired depth of workspace 60 via keyboard 26 or other input device at a later stage to permit a procedure to be carried out on workspace 60.

Alternatively, referring to FIG. 6, instrument 75 may be attached to robot arm 74. Robot arm 74 may be connected to computer 23 of CAS system 20. Computer 23 may allow robot arm 74 to be placed under substantial control of the surgeon after which robot arm 74 may be manually moved by the surgeon towards patient 22 and workspace 60 may be identified as described above with probe or instrument 75.

In optional step 112, CAS system 20 may use the information about the desired alteration location, area, or volume to simulate an appropriate alteration. Upon accepting the simulated alteration, the surgeon may use the information to provide a plan in computer 23 for altering the anatomy of patient 22. A method for simulating prosthetic implant selection and placement in an anatomical structure using a CAS system is fully described in U.S. pat. application Ser. No. 11/231,156, filed Sep. 20, 2005, entitled METHOD FOR SIMULATING PROSTHETIC IMPLANT SELECTION AND PLACEMENT, assigned to the assignee of the present application, the disclosure of which is hereby expressly incorporated herein by reference.

In step 114 and referring to FIGS. 6-7, distal tip 76 may be removed from instrument 75 and instrument 75 may be equipped with tip 77 equipped to deliver an alternative energy to workspace 60, or any other alteration location, area, or volume described herein. Instrument 75 may include a quick disconnect feature which allows a surgeon to quickly change from distal tip 76, which is used for identification purposes, to tip 77, which is used for energy delivery purposes. CAS system 20 is able to identify and/or monitor the location of tip 77, similar to identifying and/or monitoring the location of distal tip 76, because of the known geometry of instrument 75 with tip 77. In one embodiment, distal tip 76 and tip 77 have substantially the same geometry. Alternatively, distal tip 76 and tip 77 could have different geometries each of which is recognizable by CAS system 20. The surgeon may be required to input the change of tip used with instrument 75 such that CAS system 20 is aware of what is occurring. Alternatively, tip 77 may be integral with distal tip 76 such that identification of the alteration location, area, or volume and the alteration may both be done with a single tip on instrument 75, advantageously allowing the surgeon to complete the procedure without requiring a change of tips on instrument 75.

In step 116, the surgeon may grasp instrument 75, as shown in FIG. 7, and move instrument 75 towards workspace 60. As instrument 75 enters workspace 60, i.e., tip 77 of instrument 75 is near or touching bone within the boundaries of workspace 60, software 58 of computer 23 energizes alternative energy source 80.

Alternative energy source 80 may be any energy source which provides energy different from mechanical energy such as supplied to typical drill bits and cutting saw blades. For example, alternative energy source 80 may be an ultrasonic energy source, a water jet energy source, a light source such as a laser, a shock wave energy source, a vibratory energy source, or any combination thereof. Exemplary alternative energy sources 80 may be produced by S.R.A. Developments Ltd., of South Devon, United Kingdom (ultrasonic energy sources); Lumenis™ Inc., of Santa Clara, Calif. (light energy sources); Dornier MedTech, of Kennesaw, Ga. (shock wave energy sources); Plexus Technology Group Inc., of Neenah, Wis. and Ethicon Endo-Surgery, of Cincinnati, Ohio (ultrasonic vibratory sources). Some of these energy sources allow tip 77 of instrument 75 to never be required to touch any bone or soft tissue surface of an anatomical structure, and, instead, may allow the energy to be projected from tip 77 towards the anatomical structure. This projection of energy can be focused a defined distance from tip 77 so that computer 23 can precisely monitor where the action is taking place.

Also, alternative energy sources 80 may also allow alteration of soft tissue or bone without ever requiring an invasive procedure. For example, a laser may be tuned to project through tissues without harming the tissues and only have the capability to alter bone. Also, alternative energy sources 80 may be combined to work together either as at least two identical energy sources 80 or at least two non-identical energy sources 80. For example, if more than one identical energy source 80 was used, each energy source 80 by itself is not sufficient to alter any tissue or bone, but, when combined with the second (or third, fourth, etc.) identical energy source 80 focused to a predetermined known location, alteration of tissue or bone is possible. In another example, if two non-identical energy sources 80 were used, one energy source 80, e.g., a laser, may be used to alter the tissue or bone, and a second energy source 80, e.g., a water jet, may be used to remove the removed tissue or bone.

In one embodiment, once tip 77 is near or touching bone within the boundaries of workspace 60, software 58 enables alternative energy source 80 to be energized, i.e., instrument 75 is switched from a non-enabled condition to an enabled condition. In one embodiment, the surgeon may then activate actuation interface 78, e.g., a trigger or button, to cause energy to be supplied to the body of patient 22 (FIG. 2) from alternative energy source 80. When instrument 75 is in the non-enabled condition, actuation interface 78 is inoperable and, even if actuated, will not cause energy to be supplied from energy source 80. Once instrument 75 is enabled, the surgeon can selectively determine when energy is to be supplied to the body of patient 22 (FIG. 2) by activating actuation interface 78.

In one embodiment, instrument 75 must be sufficiently close to the bone to permit energy from energy source 80 to reach the bone, the closeness of which depends upon the particular energy source 80 utilized. Alternative energy source 80 is connected to computer 23 via connection 82. Connection 82 may be a hardwired connection or may be a wireless connection. Computer 23 may be connected to instrument 75 via connection 79 which may be a hardwired or wireless connection. If connection 79 is a wireless connection, instrument 75 may be provided with a plurality of reference devices 50 (FIGS. 2 and 5) to ensure that computer 23 can monitor and/or identify where instrument 75 is in relation to patient 22 (FIG. 2). Similarly, alternative energy source 80 may be connected to instrument 75 via connection 81. Connection 81 may be chosen depending on the type of alternative energy used in a desired application, as described further below.

In step 118, if the surgeon moves instrument 75 outside the bounds of workspace 60, or, if workspace 60 is a volume, beyond the three-dimensional boundary of workspace 60, e.g., instrument 75 deviates from workspace 60, computer 23 immediately de-energizes alternative energy source 80. Advantageously, upon de-energization, all emission of energy from tip 77 is immediately terminated to eliminate the potential for surrounding bone or tissue to be contacted or otherwise exposed to energy emitted from tip 77 after alternative energy source 80 is de-energized. In one embodiment, controller 25 (FIG. 3), which may take the form of a switching device, may be provided and may either be integrated within alternative energy source 80 (FIG. 3), or, alternatively, integrated within computer 23 or separated from both computer 23 and alternative energy source 80. Controller 25 may be operatively connected to computer 23 via a connection similar to connection 81 or 82, described above.

Alternatively, in step 116, instrument 75 may be guided by robot arm 74, shown in FIG. 6. Robot arm 74 is connected to computer 23 which in turn is connected to alternative energy source 80 via connection 82. Alternative energy source 80 is connected to instrument 75 via connection 81. In this manner, instrument 75 is energized when robot arm 74 moves instrument 75 into workspace 60 and tip 77 supplies the energy necessary to resect workspace 60. If, for some reason, instrument 75 moves outside the bounds of workspace 60, or, if workspace 60 is a volume, beyond the three-dimensional boundary of workspace 60, e.g., if the entire apparatus is accidentally moved or the robot malfunctions to cause instrument 75 to deviate from workspace 60, computer 23 immediately de-energizes alternative energy source 80. Advantageously, upon de-energization, all emission of energy from tip 77 is immediately terminated to eliminate the potential for surrounding bone or tissue to be contacted or otherwise exposed to energy emitted from tip 77 after alternative energy source 80 is de-energized. Alternatively, instrument 75 may be guided by haptic device 74 which provides tactile feedback to a surgeon while still maintaining control with computer 23. Both the robot arm and the haptic device may be used to offer a secondary level of accuracy to the surgeon during the procedure. For example, the robot or haptic device may be accurate to within 0.75 mm or 0.50 mm whereas the energy shutoff may be accurate to within 0.10 mm.

Once workspace 60 or any other alteration location, area, or volume is altered to a desired extent, the surgeon may complete the surgery, if necessary, by implanting a prosthetic implant. One such implant is a formable implant which is fully described in U.S. pat. application Ser. No. 11/251,181, filed Oct. 13, 2005, titled METHOD FOR REPAIRING BONE DEFECT USING A FORMABLE IMPLANT WHICH HARDENS IN VIVO, assigned to the assignee of the present application, the disclosure of which is hereby expressly incorporated herein by reference. Alternatively, once bonding or cauterizing is complete, the surgery is complete. Advantageously, alternative energy source 80 permits some surgeries to be completed with either a minimally invasive incision in patient 22 or, alternatively, no incision at all.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method for altering an anatomical structure of a patient using a computer assisted surgery system including a computer and an alternative energy source, the method comprising the steps of:
registering the anatomical structure of the patient with the computer;
inputting into the computer a workspace associated with the anatomical structure of the patient;
applying energy from the alternative energy source to the workspace with a surgical instrument; and
terminating immediately the application of energy under control from the computer when the surgical instrument deviates from the workspace.

2. The method of claim 1, wherein the surgical instrument comprises at least one of an ultrasonic device, a laser, a water jet instrument, a shock wave instrument, a light energy instrument, and a vibratory instrument.

3. The method of claim 1, wherein the alternative energy source comprises at least one of an ultrasonic energy source, a water jet energy source, a light energy source, a shock wave energy source, and a vibratory energy source.

4. The method of claim 1, further comprising, prior to said applying step, the step of converting the surgical instrument from a first, non-enabled condition, wherein the instrument is incapable of applying energy, to a second, enabled condition, wherein the instrument is capable of applying energy.

5. The method of claim 4, wherein said applying step further comprises activating an actuation interface to apply energy to the workspace when the instrument is in the second, enabled condition.

6. The method of claim 1, wherein said inputting step comprises at least one step of manually selecting the workspace via an input device on the computer, selecting a variety of points on the anatomical structure and computing the workspace based on the variety of points, and selecting the workspace on the anatomical structure by surveying the workspace on the anatomical structure.

7. The method of claim 1, further comprising, prior to said applying step, the step of simulating said applying step on the computer.

8. The method of claim 1, wherein the system further includes an instrument guide device associated with the computer, the guide device comprising at least one of a robotic device and a haptic device.

9. A computer assisted surgery system for altering an anatomical structure of a patient, the system comprising:
a computer including a workspace storage memory storing an identified workspace associated with at least one anatomical structure of a patient;
an alternative energy source;
a surgical instrument connected to said alternative energy source, said instrument convertible between a first, non-enabled condition associated with said instrument not being present in said workspace in which energy is not supplied to said instrument from said alternative energy source, and a second, enabled condition associated with said instrument being present in said workspace in which energy is supplied to said instrument from said alternative energy source; and
an energy source controller associated with said computer, said controller controlling conversion of said instrument from said second, enabled condition to said first, non-enabled condition to immediately terminate energy supplied to said instrument.

10. The system of claim 9, wherein said instrument includes an actuation interface, said actuation interface, when activated by a surgeon, causes emission of energy from said alternative energy source when said instrument is in said second condition.

11. The system of claim 9, wherein said instrument comprises at least one of an ultrasonic device, a laser, a water jet instrument, a shock wave instrument, a light energy instrument, and a vibratory instrument.

12. The system of claim 9, wherein said alternative energy source comprises at least one of an ultrasonic energy source, a water jet energy source, a light energy source, a shock wave energy source, and a vibratory energy source.

13. The system of claim 9, further comprising a workspace identifier capable of identifying said workspace and inputting said workspace into said workspace storage memory of said computer.

14. The system of claim 9, further comprising an instrument guide device associated with said computer, said guide device comprising at least one of a robotic device and a haptic device.

15. A computer assisted surgery system for altering an anatomical structure of a patient, the system controlling an alternative energy source, the system comprising:
a computer;
means for registering the anatomical structure of the patient with said computer;
means for identifying a workspace associated with the anatomical structure;
means for applying energy from the alternative energy source to the workspace; and
means for immediately terminating a supply of energy from the alternative energy source under control from said computer when said applying energy means deviates from the workspace.

16. The system of claim 15, wherein the alternative energy source comprises at least one of an ultrasonic energy source, a water jet energy source, a light energy source, a shock wave energy source, and a vibratory energy source.

17. The system of claim 15, wherein said applying means is operable between a first, non-enabled condition associated with said applying means not being present in the workspace in which energy is not supplied to said applying means from the alternative energy source, and a second, enabled condition associated with said applying means being present in the workspace in which energy is supplied to said applying means from the alternative energy source.

18. The system of claim 17, further comprising actuation means for actuating said applying means when said applying means is in said second condition.

19. The system of claim 15, further comprising an instrument guide device associated with said computer, said guide device comprising at least one of a robotic device and a haptic device.
